SEQUENCE LISTING (38 sequences)

SEQ ID NO: 1 - protein, 10 aa, Conus sp.
PVCWKFGCPL

SEQ ID NO: 2 - protein, 9 aa, Conus sp.
LFCWKFGCW

SEQ ID NO: 3 - protein, 9 aa, Conus sp.
PVCWKFGCW

SEQ ID NO: 4 - protein, 10 aa, Conus sp.
PVCWKFACPL

SEQ ID NO: 5 - protein, 10 aa, Conus sp.
LFCWKFGCPL

SEQ ID NO: 6 - protein, 9 aa, Conus sp.
PVCWKFCPL

SEQ ID NO: 7 - protein, 10 aa, Conus sp.
PVCWKFGCPL

SEQ ID NO: 8 - protein, 10 aa, Conus sp.
PVCWKFGCPL

SEQ ID NO: 9 - protein, 10 aa, Conus sp.
PVCWKFGCPL

SEQ ID NO: 10 - protein, 8 aa, Conus sp.
PVCWKFGC

SEQ ID NO: 11 - protein, 6 aa, Conus sp.
CWKFGC

SEQ ID NO: 12 - protein, 8 aa, Conus sp.
CWKFGCPL

SEQ ID NO: 13 - protein, 10 aa, Conus sp.
PVCWKAGCPL

SEQ ID NO: 14 - protein, 10 aa, Conus sp.
PVCWAFGCPL

SEQ ID NO: 15 - protein, 13 aa, Conus sp.
EGYKCWKFGCMPA

SEQ ID NO: 16 - protein, 8 aa, Conus sp.
WCWKFTCW

SEQ ID NO: 17 - protein, 10 aa, Conus sp.
PVCYKFGCPL

SEQ ID NO: 18 - protein, 10 aa, Conus sp.
PVCHKFGCPL

SEQ ID NO: 19 - protein, 9 aa, Conus sp.
PVCWKFSCW

SEQ ID NO: 20 - protein, 9 aa, Conus sp.
PVCWKFTCW

SEQ ID NO: 21 - protein, 9 aa, Conus sp.
PVCWKFNCW

SEQ ID NO: 22 - protein, 10 aa, Conus sp.
KVCWKFGCPL

SEQ ID NO: 23 - protein, 10 aa, Conus sp.
MVCWKFGCPL

SEQ ID NO: 24 - protein, 10 aa, Conus sp.
PVCWKFGCPL

SEQ ID NO: 25 - protein, 10 aa, Conus sp.
PDCWKFGCPL

SEQ ID NO: 26 - protein, 10 aa, Conus sp.
PPCWKFGCPL

SEQ ID NO: 27 - protein, 10 aa, Conus sp.
PPCWKFGCPL

SEQ ID NO: 28 - protein, 10 aa, Conus sp.
PVCFKFGCPL

SEQ ID NO: 29 - protein, 10 aa, Conus sp.
PVCWRFGCPL

SEQ ID NO: 30 - protein, 10 aa, Conus sp.
PVCWKFTCPL

SEQ ID NO: 31 - protein, 10 aa, Conus sp.
PVCWKFSCPL

SEQ ID NO: 32 - protein, 10 aa, Conus sp.
PVCWKFVCPL

SEQ ID NO: 33 - protein, 10 aa, Conus sp.
PVCWKFNCPL

SEQ ID NO: 34 - protein, 10 aa, Conus sp.
PVCWKFLCPL

SEQ ID NO: 35 - protein, 10 aa, Conus sp.
PVCWKFICPL

SEQ ID NO: 36 - protein, 10 aa, Conus sp.
PVCWKFGCLL

SEQ ID NO: 37 - protein, 10 aa, Conus sp.
PVCWKFGCIL

SEQ ID NO: 38 - protein, 10 aa, Conus sp.
PVCWKFGCPP

(19) United States
(12) Patent Application Publication
(10) Pub. No.: US 2025/0257098 A1
(43) Pub. Date: Aug. 14, 2025

(54) NOVEL SST4 SELECTIVE AGONISTS AS NON-OPIOID ANALGESICS

(71) Applicants: KØBENHAVNS UNIVERSITET, København K (DK); UNIVERSITY OF UTAH RESEARCH FOUNDATION, Salt Lake City, UT (US)

(72) Inventors: Walden BJØRN-YOSHIMOTO, København K (DK); Iris Bea L. RAMIRO, København K (DK); Thomas Lund KOCH, København K (DK); Helena SAFAVI-HEMAMI, København K (DK)

(73) Assignees: KØBENHAVNS UNIVERSITET, København K (DK); UNIVERSITY OF UTAH RESEARCH FOUNDATION, Salt Lake City, UT (US)

(21) Appl. No.: 18/849,884

(22) PCT Filed: Mar. 14, 2023
(86) PCT No.: PCT/EP2023/056466

§ 371 Date: Sep. 23, 2024

(30) Foreign Application Priority Data
Mar. 22, 2022 (EP) 22163579.0

(51) Int. Cl. C07K 7/56; A61K 9/00; A61K 38/12; A61P 29/00

(52) U.S. Cl. CPC C07K 7/56; A61K 9/0019; A61K 38/12; A61P 29/00

(57) ABSTRACT

The present invention relates to novel somatostatin 4 (SST4) selective agonists as non-opioid analgesics. In particular, the present invention relates to peptides having strong SST4-selectivity for use in treatment of pain or inflammation.

### SEQUENCE LISTING

The Sequence Listing is submitted as an XML file in the form of a file named 18849884_2_1.xml (33,511 bytes) created on Mar. 24, 2025, which is herein incorporated by reference in its entirety.

### FIELD OF THE INVENTION

The present invention relates to novel somatostatin receptor 4 (SST4) selective agonists as non-opioid analgesics. In particular, the present invention relates to peptides having strong SST4-selectivity for use in treatment of pain or inflammation.

The project leading to this application has received funding from the European Research Council (ERC) under the European Union's Horizon 2020 research and innovation program (grant agreement No 949830).

### BACKGROUND OF THE INVENTION

The inventors of the present invention have discovered that a largely unexplored, deep-water lineage of fish-hunting cone snails of the Asprella clade, in particular the two species Conus rolani and Conus neocostatus, uses a relatively slow predation strategy, i.e. the Asprella envenomation takes 1-3 hours from the first strike to engulfment of prey. This unexpected observation led the inventors to investigate venom components that potentially facilitate the hitherto unknown behavior observed. By combining the behavioral observations of prey-capture by the cone snail predator with behavioral changes elicited by the action of individual venom components in mice, a novel class of analogs of the vertebrate hormone somatostatin was discovered.

Further, somatostatin (SS) is a peptide hormone with diverse physiological roles. By investigating a deep-water clade of fish-hunting cone snails, the inventors showed that predator-prey evolution has generated a diverse set of SS analogs, each optimized to elicit specific systemic physiological effects in prey. The increased metabolic stability, distinct SS receptor (SST) activation profiles, and chemical diversity of the venom analogs make them suitable leads for therapeutic application, including pain and inflammation.

WO 2017/139845 A1 discloses inter alia conotoxins the context of treating pain, including chronic pain, inflammatory pain, neuropathic pain, visceral pain, breakthrough pain, bipolar disorder, Alzheimer's disease, Parkinson's disease, producing analgesia or enhancement of opiate analgesia. The conotoxins of WO 2017/139845 A1 are not disclosed in the context of SST4 agonist properties.

Olivera B. M. (Annual report dated October 2020, prepared for the U.S. Army Medical Research and Development Command Fort Detrick, Maryland 21702-5012 “Novel Strategies for Accelerating Non-Opioid Drug Discovery” University of Utah) discloses e.g. venoms collected from Conus rolani, including peptides, such as Cont-Ro4, shown to induce analgesia in the formalin pain model.

While opioids are very efficient in the management of acute nociceptive pain, they fall short with chronic pain. Additionally, opioids have the disastrous pharmacologic profile of dependence after prolonged use and carry a high risk of respiratory depression.

Hence, the provision of improved non-opioid based analgesic having their effect outside of the brain would be highly advantageous.

### SUMMARY OF THE INVENTION

The invention relates to peptides from cone snails, hereinafter merely referred to as peptides, that activate a certain type of somatostatin receptor (mainly SST4), and are some of the most, if not the most selective compounds ever described for this receptor.

The peptides of the invention provide analgesia that is (1) via a non-opioid based mechanism and (2) by actions outside of the central nervous system. The peptides of the invention are intended for pain management, where they can alleviate pain, while avoiding the addictive properties and/or side effects of the current gold standards, such as opioids or anticonvulsants. The peptides of the invention are also intended for use in the treatment of inflammation.

Activation of SST4, and to some degree somatostatin receptor 1 (SST1) with the peptides of the present invention, has been shown to have analgesic effects, as well as anti-inflammatory effects.

The inventors recently described a novel class of cyclical somatostatin receptor activating peptides. One of those identified, consomatin Ro1 (also referred to as “Cont-Ro1”), showed a low potency SST1 and SST4 (˜5 μM) activation and analgesic effects as strong as morphine, but longer lasting, when injected peripherally in mice. Notably, this only occurred when injected peripherally, not when injected intracranially, suggesting a non-brain-based target, and suggesting that the peptide does not have to cross the blood-brain-barrier to induce analgesia.

Based on the Cont-Ro1 findings, the inventors looked for additional “somatostatin-like” peptides, and found e.g., one of the peptides this patent application concerns, here called HSH-109. Initial tests showed it to be (A) more potent and (B) more selective for SST4 than Cont-Ro1 (concentration-response curve shown under “5. Developmental stage”). The apparent potencies observed for the initial peptide (HSH-109) are in the low nanomolar range for SST4, and in the low micromolar range for SST1. Interestingly, the peptide did not significantly activate the SST2, SST3 or SST5 at these concentrations.

Thus, one aspect of the invention relates to a peptide comprising or consisting of a sequence of the following consecutive amino acids: cysteine, D-tryptophan, lysine, phenylalanine, glycine, and cysteine, wherein the first indicated cysteine residue makes disulfide bonds to the last indicated cysteine residue.

Another aspect of the present invention relates to a peptide comprising or consisting of the amino acid sequence:
Xaa1, Xaa2, Xaa3, Xaa4, Xaa5, Xaa6 Xaa7, Xaa8, Xaa9 and Xaa10, wherein
Xaa1 is selected from proline, leucine, tyrosine, lysine, methionine, or is absent;
Xaa2 is selected from valine, phenylalanine, lysine, tryptophan, aspartate, proline, or is absent;
Xaa3 is cysteine;
Xaa4 is tryptophan, tyrosine, phenylalanine, or histidine;
Xaa5 is lysine or arginine;
Xaa6 is phenylalanine;
Xaa7 is glycine, alanine, threonine, serine, valine, asparagine, leucine, isoleucine, or is absent;
Xaa8 is cysteine;
Xaa9 is proline, tryptophan, methionine, leucine, isoleucine, or is absent;
Xaa10 is leucine, proline, or is absent, wherein the cysteine in Xaa3 is (i) cysteine making disulfide bonds to the cysteine of Xaa8 or (ii) is replaced by other amino acids that can form bonds to Xaa8.

Yet another aspect of the present invention is to provide a peptide according to the present invention, for use as a medicament in a human or mammal subject and/or for use in treatment of pain or inflammation in a human or mammal subject.

Still another aspect of the present invention relates to the use of an isolated peptide selected from the group consisting of:
(a) peptides comprising amino acid sequences set forth in SEQ ID NOs: 1-38; or
(b) peptides having at least 80% sequence identity to any of the amino acid sequences set forth in SEQ ID NOs: 1-38; or
(c) peptides having at least 80% sequence identity to the conserved core sequence consisting of the following consecutive amino acids: cysteine, D-tryptophan, lysine, phenylalanine, glycine and cysteine (CwKFGC),
for use as a non-opioid analgesic or an anti-inflammatory agent.

### BRIEF DESCRIPTION OF THE FIGURES

FIG. 1 shows representative concentration-response curves of human somatostatin-14 (SS-14) at the five human somatostatin receptors (SST1-5) in the PRESTO-Tango β-arrestin recruitment assay (a standardized GPCR activation assay). Each curve is normalized to maximum somatostatin response at the given receptor. EC₅₀ values are shown in FIG. 7.

FIG. 2 shows representative concentration-response curves of HSH-109 (SEQ ID NO 1) at the five human somatostatin receptors (SST1-5) in the PRESTO-Tango β-arrestin recruitment assay (a standardized GPCR activation assay). Each curve is normalized to maximum somatostatin response. EC₅₀ values are shown in FIG. 7.

FIG. 3 shows representative concentration-response curves of HSH-133 (SEQ ID NO 2) at the five human somatostatin receptors (SST1-5) in the PRESTO-Tango β-arrestin recruitment assay (a standardized GPCR activation assay). Each curve is normalized to maximum somatostatin response. EC₅₀ values are shown in FIG. 7.

FIG. 4 shows representative concentration-response curves of HSH-142 (SEQ ID NO 3) at the five human somatostatin receptors (SST1-5) in the PRESTO-Tango β-arrestin recruitment assay (a standardized GPCR activation assay). Each curve is normalized to maximum somatostatin response. EC₅₀ values are shown in FIG. 7.

FIG. 5 shows the effect on mechanical hypersensitivity of a single injection of 0.004-2.5 mg/kg of HSH-109 in a post-surgical pain model in mice using normal saline as a negative control, and 5 mg/kg morphine as a positive control. FIG. 5 is response over time post-surgery with compound injection occurring immediately after the “Post” point. Six mice were used in each condition, and the datapoints represent mean values, with the error bars being standard deviation.

FIG. 6 shows the effect of a single injection of 0.004-2.5 mg/kg of HSH-109 in a post-surgical pain model in mice (Von Frey hair measurement) using normal saline as a negative control, and 5 mg/kg morphine as a positive control. FIG. 6 is a summary of the area under the curve (AUC) of data shown in FIG. 5, showing each mouse as a single datapoint in the overlayed scatterplot. Six mice were used in each condition, the bars show the mean, and the error bars represent 95% confidence intervals. Kruskal-Wallis test with Dunn's post-test was performed for statistical comparisons. * indicates p<0.05, indicates P<0.01.

FIG. 7 shows a heatmap of the mean EC₅₀ values of each compound tested at the five somatostatin receptors in the PRESTO-Tango β-arrestin recruitment assay (a standardized GPCR activation assay). The top is somatostatin-14, bottom is the SST4 selective small molecule, J-2156. All peptides are labelled with their name and amino acid sequence in parenthesis. Lower case letters denote D-amino acids, “O” indicates hydroxyproline. An “X” indicates that the curve was not completed at the highest concentration used, and thus a reliable EC₅₀ value could not be extracted, indicating that the compound was less potency at a given receptor, than at receptors with a listed EC₅₀ value.

FIG. 8 shows a heatmap of the mean EC₅₀ values of each compound tested at the five somatostatin receptors in the PRESTO-Tango β-arrestin recruitment assay (a standardized GPCR activation assay), similar to FIG. 7. All peptides are labelled with their name and 20 amino acid sequence in parenthesis. Lower case letters denote D-amino acids, “O” indicates hydroxyproline. An “X” indicates that the curve was not completed at the highest concentration used, and thus a reliable EC₅₀ value could not be extracted, indicating that the compound was less potent at a given receptor, than at receptors with a listed EC₅₀ value.

FIG. 9 (chronic neuropathic pain) shows the effect of compounds in a model of chronic neuropathic pain (spared nerve injury, SNI) as measured by von Frey hairs. Surgery to restrict the sciatic nerve was performed on one side (ipsilateral), and the neuropathic pain was allowed to develop. On day seven, a post-surgery baseline measurement was performed, and on day 16 a new baseline measurement is performed, followed by administration of saline, either 0.5 or 5.0 mg/kg HSH-109, or 30 mg/kg gabapentin intraperitoneally and measurements at indicated times. Dotted lines show response on the contralateral side, and the full lines show the same on the ipsilateral side. Five (gabapentin) or six (all other conditions) male mice per group were used, and the datapoints represent mean values, with the error bars being standard deviation. Experimenters were blinded.

FIG. 10 (inflammatory pain) shows the effect of compounds in a model of inflammatory pain (complete Freund's adjuvant, CFA) as measured by von Frey hairs. CFA was administered by intraplantar injection. Two days later a baseline measurement was performed, followed by administration of saline, or 0.1 mg/kg of either HSH-109, HSH-152, and HSH-201 (sequences in FIGS. 7 and 8) intraperitoneally and measurements at indicated times. The following day a baseline measurement was performed, followed by administration of saline, or 0.25 mg/kg of either HSH-109, HSH-152, and HSH-201 intraperitoneally and measurements at indicated times. Seven male mice per group were used, and the datapoints represent mean values, with the error bars being standard deviation. Experimenters were blinded.

FIG. 11 shows the effect of 10 nM of HSH-109 in a model of lipopolysaccharide (LPS)-stimulated inflammation in mouse macrophages, as assessed by ELISA analysis of the inflammatory markers tumor necrosis factor (TNFα) and monocyte chemoattractant protein 1 (MCP1). Data shows the level of TNFα and MCP1 as percent of LPS-stimulated, non HSH-109 treated. Bars show the average of two wells, and error bars represent standard deviations.

FIG. 12 shows a comparison of 2.5 mg/kg of Cont-Ro1 and HSH-109. Both experiments were performed in the same lab according to FIG. 5 (HSH-109 data is a normalized data from 2.5 mg/kg from FIG. 5). Data is normalized to showing percentage of pre-surgery thresholds of force applied using von Frey hairs.

FIG. 13 shows the area under the curve (AUC) of the data shown in FIG. 12.

The present invention will now be described in more detail in the following.

### DETAILED DESCRIPTION OF THE INVENTION

Prior to discussing the present invention in further details, the following terms and conventions will first be defined:

### Embodiments

In one embodiment of the present invention there is provided a peptide comprising or consisting of a sequence of the following consecutive amino acids: cysteine, D-tryptophan, lysine, phenylalanine, glycine, and cysteine, wherein the first indicated cysteine residue makes disulfide bonds to the last indicated cysteine residue.

In another embodiment of the present invention there is provided a peptide comprising or consisting of a sequence of the following consecutive amino acids: cysteine, D-tryptophan, lysine, phenylalanine, glycine, and cysteine, wherein the first indicated cysteine residue makes disulfide bonds to the last indicated cysteine residue; for use as a medicament.

In still another embodiment of the present invention there is provided a peptide comprising or consisting of a sequence of the following consecutive amino acids: cysteine, D-tryptophan, lysine, phenylalanine, glycine, and cysteine, wherein the first indicated cysteine residue makes disulfide bonds to the last indicated cysteine residue; for use in treatment of pain or inflammation in a human or mammal subject.

In still another embodiment of the present invention there is provided a peptide comprising or consisting of an amino acid sequence of SEQ ID NO 1-38.

In still another embodiment of the present invention there is provided a peptide comprising or consisting of an amino acid sequence of SEQ ID NO 1-38, for use as a medicament.

In still another embodiment of the present invention there is provided a peptide comprising or consisting of an amino acid sequence of SEQ ID NO 1-38, for use in treatment of pain or inflammation in a human or mammal subject.

In still another embodiment of the present invention there is provided a peptide comprising or consisting of the amino acid sequence:
Xaa1, Xaa2, Xaa3, Xaa4, Xaa5, Xaa6 Xaa7, Xaa8, Xaa9 and Xaa10, wherein
Xaa1 is selected from proline, leucine, tyrosine, lysine, methionine, hydroxyproline, or is absent;
Xaa2 is selected from valine, phenylalanine, lysine, tryptophan, aspartate, proline, hydroxyproline, or is absent;
Xaa3 is cysteine or (i) cysteine making disulfide bonds to the cysteine of Xaa8 or (ii) is replaced by an amino acid that can form dicarba bonds, other amino acids, or penicillamine that can form bonds to Xaa8;
Xaa4 is tryptophan, tyrosine, phenylalanine, or histidine;
Xaa5 is lysine or arginine;
Xaa6 is phenylalanine or D-phenylalanine;
Xaa7 is glycine, alanine, threonine, serine, valine, asparagine, leucine, isoleucine, or is absent;
Xaa8 is cysteine or (i) cysteine making disulfide bonds to the cysteine of Xaa8 or (ii) is replaced by an amino acid that can form dicarba bonds, other amino acids, or penicillamine that can form bonds to Xaa3;
Xaa9 is proline, hydroxyproline, tryptophan, methionine, leucine, isoleucine, or is absent;
Xaa10 is leucine, proline, hydroxyproline, or is absent.

In still another embodiment of the present invention there is provided a peptide comprising or consisting of the amino acid sequence:
Xaa1, Xaa2, Xaa3, Xaa4, Xaa5, Xaa6 Xaa7, Xaa8, Xaa9 and Xaa10, wherein
Xaa1 is selected from proline, leucine, tyrosine, lysine, methionine, hydroxyproline, or is absent;
Xaa2 is selected from valine, phenylalanine, lysine, tryptophan, aspartate, proline, hydroxyproline, or is absent;
Xaa3 is cysteine or (i) cysteine making disulfide bonds to the cysteine of Xaa8 or (ii) is replaced by an amino acid that can form dicarba bonds, other amino acids, or penicillamine that can form bonds to Xaa8;
Xaa4 is tryptophan, tyrosine, phenylalanine, or histidine;
Xaa5 is lysine or arginine;
Xaa6 is phenylalanine or D-phenylalanine;
Xaa7 is glycine, alanine, threonine, serine, valine, asparagine, leucine, isoleucine, or is absent;
Xaa8 is cysteine or (i) cysteine making disulfide bonds to the cysteine of Xaa8 or (ii) is replaced by an amino acid that can form dicarba bonds, other amino acids, or penicillamine that can form bonds to Xaa3;
Xaa9 is proline, hydroxyproline, tryptophan, methionine, leucine, isoleucine, or is absent;
Xaa10 is leucine, proline, hydroxyproline, or is absent;
for use as a medicament.

In still another embodiment of the present invention there is provided a peptide comprising or consisting of the amino acid sequence:
Xaa1, Xaa2, Xaa3, Xaa4, Xaa5, Xaa6 Xaa7, Xaa8, Xaa9 and Xaa10, wherein
Xaa1 is selected from proline, leucine, tyrosine, lysine, methionine, hydroxyproline, or is absent;
Xaa2 is selected from valine, phenylalanine, lysine, tryptophan, aspartate, proline, hydroxyproline, or is absent;
Xaa3 is cysteine or (i) cysteine making disulfide bonds to the cysteine of Xaa8 or (ii) is replaced by an amino acid that can form dicarba bonds, other amino acids, or penicillamine that can form bonds to Xaa8;
Xaa4 is tryptophan, tyrosine, phenylalanine, or histidine;
Xaa5 is lysine or arginine;
Xaa6 is phenylalanine or D-phenylalanine;
Xaa7 is glycine, alanine, threonine, serine, valine, asparagine, leucine, isoleucine, or is absent;
Xaa8 is cysteine or (i) cysteine making disulfide bonds to the cysteine of Xaa8 or (ii) is replaced by an amino acid that can form dicarba bonds, other amino acids, or penicillamine that can form bonds to Xaa3;
Xaa9 is proline, hydroxyproline, tryptophan, methionine, leucine, isoleucine, or is absent;
Xaa10 is leucine, proline, hydroxyproline, or is absent;
for use in treatment of pain or inflammation in a human or mammal subject.

In still another embodiment of the present invention there is provided a method of treating or prevention of pain, such as acute, post-surgical, inflammatory, chronic, visceral or breakthrough pain, comprising administering to a subject in need thereof an effective amount of a peptide according to the present invention.

In still another embodiment, the present invention provides a method for the treatment or prevention of inflammation comprising administering to a subject in need thereof an effective amount of a peptide according to the present invention.

In still another embodiment, the present invention provides An isolated peptide selected from the group consisting of:
(a) peptides comprising amino acid sequences set forth in SEQ ID NOs: 1-38; or
(b) peptides having at least 80% sequence identity to any of the amino acid sequences set forth in SEQ ID NOs: 1-38; or
(c) peptides having at least 80% sequence identity to the conserved core sequence consisting of the following consecutive amino acids: cysteine, D-tryptophan, lysine, phenylalanine, glycine and cysteine (CwKFGC),
for use as a non-opioid analgesic or an anti-inflammatory agent.

While the peptide according to the invention may be the sole active ingredient administered to the subject, the administration of other active ingredients with said peptide is within the scope of the invention. For example, the peptide could be administered with one or more therapeutic agents.

The peptides according to the present invention can be combined with other treatment options known to be used in the art in connection with a treatment of pain or inflammation, including inter alia combinations with non-limiting list of medicaments: non-steroidal anti-inflammatory drugs (NSAIDs) including COX-2 inhibitors; opiate receptor agonists; cannabinoid receptor modulators; sodium channel blockers; N-type calcium channel blockers; serotonergic and noradrenergic modulators; corticosteroids; histamine H1, H2, H3 and H4 receptor antagonists; proton pump inhibitors; leukotriene antagonists and 5-lipoxygenase inhibitors; local anesthetics; VR1 agonists and antagonists; nicotinic acetylcholine receptor agonists; P2X3 receptor antagonists; NGF agonists and antagonists or anti-NGF antibodies; NK1 and NK2 antagonists; Bradykinin B1 antagonists; CCR2 antagonists; iNOS or nNOS or eNOS inhibitors; NMDA antagonist; potassium channel modulators; GABA modulators; serotonergic and noradrenergic modulators; anti-migraine drugs; neuropathic pain drugs such as pregabaline or duloxetine.

As will be readily appreciated by those skilled in the art, the route of administration will depend on the nature of the condition and the human or mammal to be treated. As is generally known in the art, in the preparation of any formulation containing the peptide actives, care should be taken to ensure that the activity of the peptide is not destroyed in the process and that the peptide is able to reach its site of action without being destroyed. Also, as is well known in the art some formulations are given to enhance the activity, e.g., by improve the availability or increase the solubility of a compound. In some circumstances it may be necessary to protect the peptide by means known in the art, such as, for example, micro encapsulation. Similarly, the route of administration chosen should be such that the peptide reaches its site of action.

Another aspect of the present invention relates to prodrugs which may be metabolized to any of the peptides of the invention (SEQ ID NOs 1-38) once taken up into the body.

In some embodiments, a peptide or a composition comprising a peptide of the invention may be administered orally, dermally, topically, or subcutaneously.

In some embodiments, a peptide or a composition comprising a peptide of the invention may be administered in doses of 0.5-10 mg/kg, such as 2.5 mg/kg (mice) or 0.1-1 mg/kg (humans). Other dosages could be envisaged.

### Definitions

Amino acid

The term “amino acid” is used herein in its broadest sense and may refer to compounds having an amino group and a carboxylic acid group. The amino acids incorporated into the peptides of the present invention may be D- or L-forms of proteogenic or naturally occurring amino acids, or may be D- or L-forms of non-proteogenic or non-naturally occurring amino acids. As referred to herein, the term extends to synthetic amino acids and analogues thereof, including salts, isomers, tautomers, esters, and N-methylated amino acids.

### Peptide or Protein

The term “peptide” as used herein in relation to the invention refers to peptides comprising a conserved core sequence consisting of the following consecutive amino acids: cysteine, D-tryptophan, lysine, phenylalanine, glycine and cysteine (CwKFGC), including e.g. the peptide HSH-109 found in snails of the genus Conus. Since both modifications and exact cleavage sites for the mature peptides are not readily predictable from the DNA or RNA sequence, “peptide” is used to cover any cleavage pattern or naturally occurring posttranslational modification that can arise, given an RNA or DNA sequence. The terms “peptide” and “protein” are used herein interchangeably in their broadest sense to refer to oligomers of two or more amino acids, including isolated, synthetic, or recombinant peptides. These terms apply to amino acid polymers in which one or more amino acid residues is/are a synthetic non-naturally occurring amino acid, such as a chemical analogue of a corresponding naturally occurring amino acid, as well as to naturally occurring amino acid polymers. These terms do not exclude modifications, for example, glycosylations, acetylations, phosphorylations and the like. Soluble forms of the subject peptides are particularly useful. Included within the definition are, for example, peptides containing one or more analogs of an amino acid including, for example, unnatural amino acids or peptides with substituted linkages.

### Consomatin Ro1 (Cont-Ro1)

Consomatin Ro1 is a venom peptide and as used herein refers to a mimetic of the vertebrate hormone somatostatin. Consomatin Ro1 represents the first SS-like peptide that was isolated from cone snail venom.

### Consomatin

The term consomatin as used herein refers to any peptide, as defined above, that belongs to the so-called C-toxin gene superfamily and mimics the sequence of somatostatin by having two cysteines that form a disulfide bond and a tryptophan-lysine (WK) motif between the two cysteines that form the disulfide.

### Exon Capture

When used herein “exon capture” refers to a method of sequencing the coding regions of a genome.

### Pain

When used herein “pain” refers to and encompasses, but is not limited to, inflammatory pain, neuropathic pain, chronic pain, visceral pain, breakthrough pain, acute pain, nociceptive pain, and radicular pain.

### Somatostatin (SS)

SS is a highly conserved vertebrate peptide hormone that, in humans, is predominantly secreted in the brain, pancreas, and gastrointestinal tract where it functions as an inhibitor of secretion and cell proliferation. SS was first discovered as the main inhibitor of growth hormone (GH)-release from the anterior pituitary gland and has since been associated with many additional biological functions, including the inhibition of pancreatic hormone secretion, neuronal signaling, pain, and inflammation.

### SST4 (or SST1) “Selective” and “Selectivity”

The terms “selective” and “selectivity” as used herein refers to an activation on one subtype of receptor (e.g., SST4) with a potency at least 30-fold higher than for the subtype it displays “selectivity” over. If not compared explicitly to any one specific somatostatin receptor, it is assumed that it is compared to all other human somatostatin receptor subtypes.

### EXAMPLES

### Example 1—Peptide Synthesis and Purification

The discovery of Cont-Ro1 led the inventors to search for other SS-like peptides in cone snail venom.

In order to specifically identify candidates that likely activate the SST, the inventors searched for sequences that belong to the C-toxin gene family and had at least five amino acid residues that are known to be important for SST activation by human somatostatin. These amino acids included two cysteines, a phenylalanine, a tryptophan, and a lysine. The inventors searched cone snail transcriptome, genome and exon capture data and identified a DNA sequence from cone snail exon capture data that they performed initial in vitro tests on.

The inventors have also designed the first round of analogs based on substituting amino acid residues found in other peptides in the conopeptide inventory and performed initial tests of the first of these analogs.

The peptides according to the present invention may be prepared using standard peptide synthetic methods followed by oxidative disulfide bond formation, for example as discussed in the present Example.

### Peptide Synthesis and Purification

HSH-109 and HSH-109 analogs were synthesized using solid phase peptide synthesis on an automated peptide synthesizer. Peptides were cleaved from resins and oxidized in the presence of dimethylsulfoxide (DMSO). Folded peptides were purified using high-performance liquid chromatography (HPLC) and dried in a lyophilizer for storage.

### Example 2—Concentration-Response

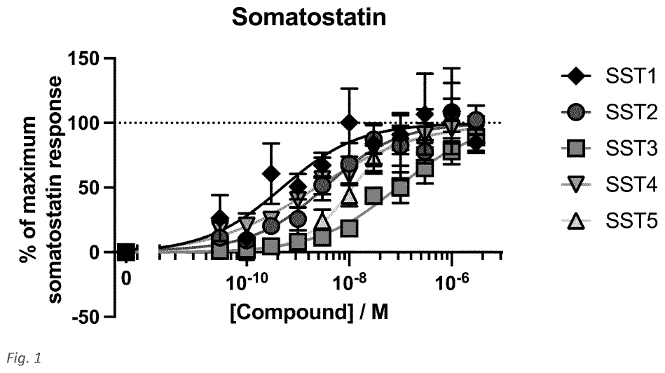

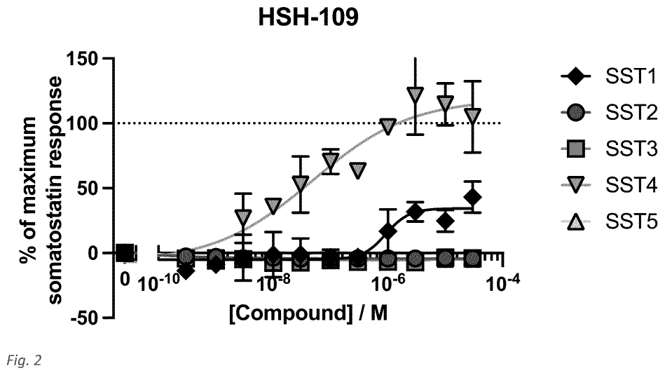

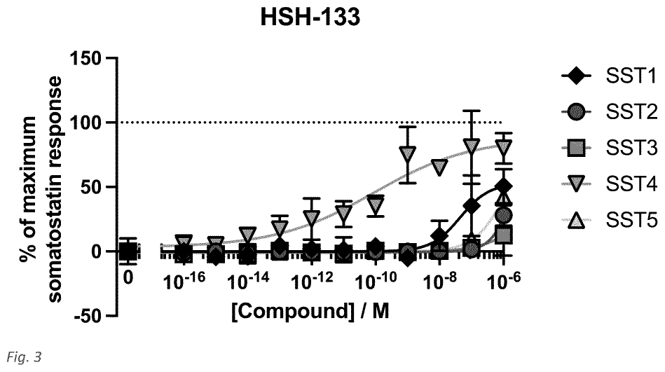

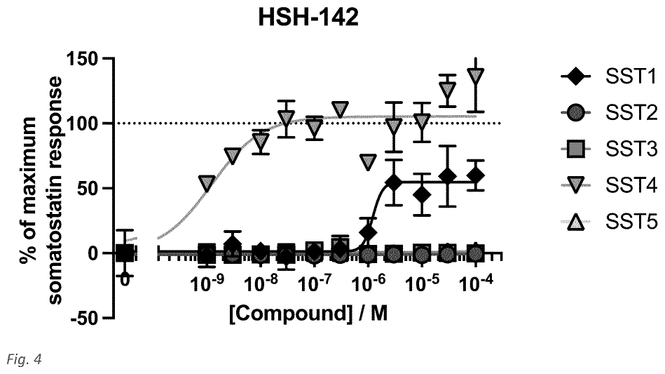

Concentration-response curves of somatostatin-14 (SS-14), HSH-109, HSH-133, and HSH-142 at the five human somatostatin receptors (SST1-5) in the PRESTO-Tango β-arrestin recruitment assay (a standardized GPCR activation assay) are shown in FIGS. 1-4.

HTLA cells were maintained in Dulbecco's modified Eagle's medium (DMEM) supplemented with 10% fetal bovine serum (FBS), penicillin (100 U/ml), and streptomycin (100 μg/ml) (HTLA medium), with hygromycin B (100 μg/ml) and puromycin (2 μg/ml) added. On day 1, 3 million cells were seeded in T25 flasks in 5 ml of HTLA medium and incubated overnight. On day 2, medium was changed, and cells were transfected using PolyFect according to the manufacturer's protocol. On day 3, the cells were resuspended in DMEM supplemented with 1% defined FBS (assay medium), and 15,000 cells in 40 μl per well were seeded in poly-d-lysine-coated white clear-bottom 384-well plates and incubated overnight. On day 4, medium was changed, and ligands were diluted to appropriate concentrations (5× final concentration) in Hanks' balanced salt solution (HBSS) supplemented with 20 mM HEPES, 1 mM CaCl2, and 1 mM MgCl2, pH adjusted to 7.4 with 10 M NaOH (assay buffer), which was supplemented with 0.1% bovine serum albumin (ligand buffer). Ten microliters per well was added, and cells were incubated overnight. On day 5, the medium and compounds were removed from the cells, and 20 μl per well of a 1:20 dilution of Bright-Glo (Promega) in assay buffer supplemented with 0.01% Pluronic F68 was added to the cells. Plates were incubated for 20 min in the dark at room temperature, and luminescence was measured on Molecular Devices SpectraMax iD5 plate reader with each well integrated for 1 s.

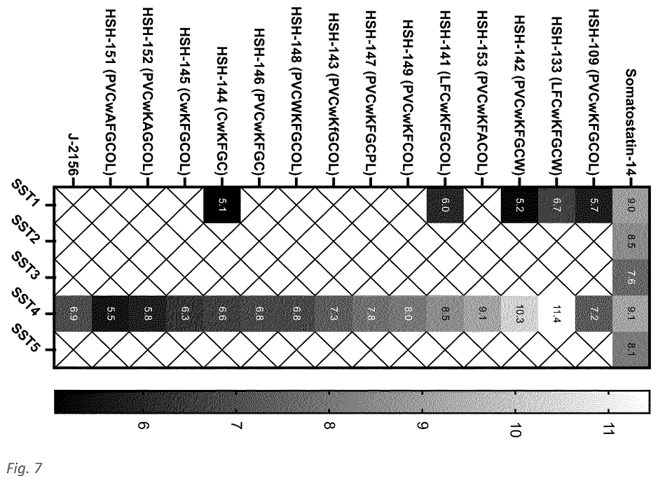

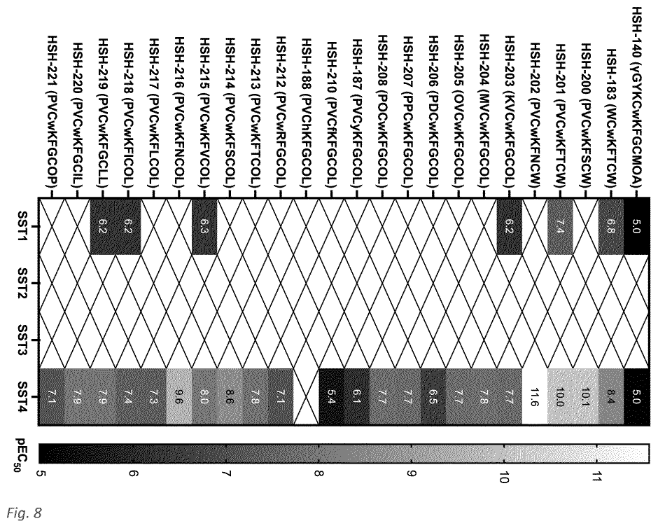

Data from this setup is presented in FIGS. 1-4, 7, and 8. The inventors observed a potency and selectivity range at the SST4 showing the breadth of modifications permissible (FIGS. 7 and 8).

### Example 3—Model of Acute Post-Surgical Pain

This experiment was set up to determine the effect of compounds in a model of acute post-surgical pain as measured by mechanical hypersensitivity.

### Paw Incision Model of Post-Surgical Pain

To analyze post-surgery acute incisional hypersensitivity, a plantar incision model was used as described by Brennan et al. (Brennan T J, Zahn P K, Pogatzki-Zahn E M. Mechanisms of incisional pain. Anesthesiol Clin North Am 2005; 23(1):1-20). Briefly, a 0.5 cm long incision, from the heel toward the toes, was made through skin and fascia of the plantar aspect of the left hind paw, including the underlying muscle. The plantaris muscle was then elevated and longitudinally incised, leaving the muscle origin and insertion intact. After hemostasis with gentle pressure, the skin was closed with two mattress sutures of 5-0 nylon on a curved needle.

### Intraperitoneal Injections

24 hours post-surgery the injection site (left lower abdominal quadrant) was wiped with 2% chlorhexidine. Mice were manually restrained, abdomen side up, with cranial end pointed down. With an angle of 15° to 20°, the abdominal cavity was punctured to inject either 200 μl of indicated doses of indicated compounds, or vehicle (normal saline or PBS, as indicated). Before injection, aspiration was attempted to ensure that an abdominal viscus had not been penetrated.

### Measurement of Tactile Sensory Thresholds

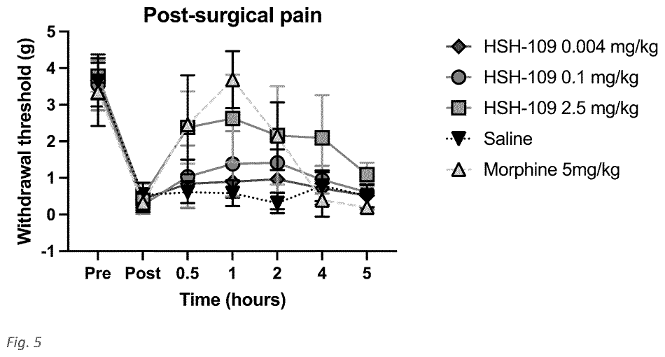

The assessment of tactile sensory thresholds was determined by measuring the withdrawal response to probing the plantar surface of the hind paw with a series of calibrated fine filaments (von Frey) at time points indicated in FIG. 5. Each filament was applied perpendicularly to the plantar surface of the paw of mice held in suspended wire mesh cages. The “up and down” method was used to identify the mechanical force required for a paw withdrawal response. The size range of stimuli was between 2.44 (0.4 milliNewtons) and 4.56 (39.2 mN). The starting filament is 3.61 (3.9 mN). The filament was placed perpendicularly to the skin with slowly increasing force until it bends. It stays bent for approximately 1 second and was then removed. Data were analyzed with the nonparametric method of Dixon, as described by Chaplan et al. (S. R. Chaplan, F. W. Bach, J. W. Pogrel, J. M. Chung, T. L. Yaksh, Quantitative assessment of tactile allodynia in the rat paw. Journal of neuroscience methods 53, 55-63 40 (1994).). Results are expressed as the mean withdrawal threshold that induces a paw withdrawal response in 50% of animals.

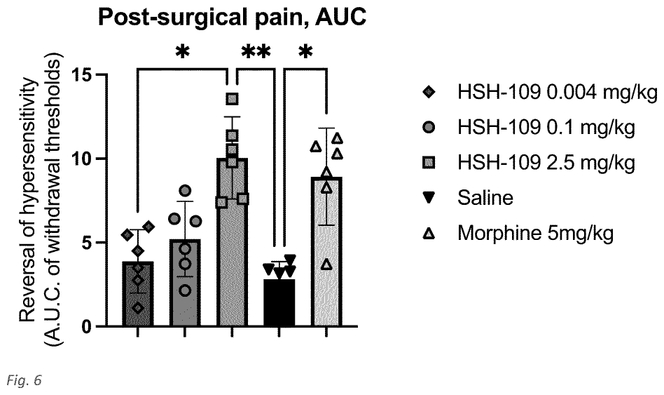

Data from this setup is presented in FIGS. 5 and 6.

The inventors observed a robust reduction of hypersensitivity at 2.5 mg/kg in the mouse model of post-surgical pain, which was longer lasting than the morphine positive control. At present, it is not clear whether saturating doses was used or if higher dose would give increased effect. Expected doses for mice would be 0.5-10 mg/kg and for humans 0.1-1 mg/kg if injected.

### Example 4—Model of Chronic Neuropathic Pain (Spared Nerve Injury, SNI)

This experiment was set up to determine the effect of compounds in a model of chronic neuropathic pain (spared nerve injury, SNI) as measured by mechanical hypersensitivity.

### SNI Surgery

SNI surgery was performed on the left hindleg of the C57BL/6NRj mice under 2% isoflurane anaesthesia. Skin on the lateral surface was incised between hip and knee followed by lengthwise division of the biceps femoris muscle leading to exposure of the three branches of the sciatic nerve. Sural branch was left intact, while the peroneal and tibial branches were ligated with a single surgical knot and axotomized distally of the ligation. Wounds were closed with surgical glue and animal were monitored daily for signs of stress or discomfort, but in all cases recovered uneventfully.

### Measurement of Tactile Sensory Thresholds

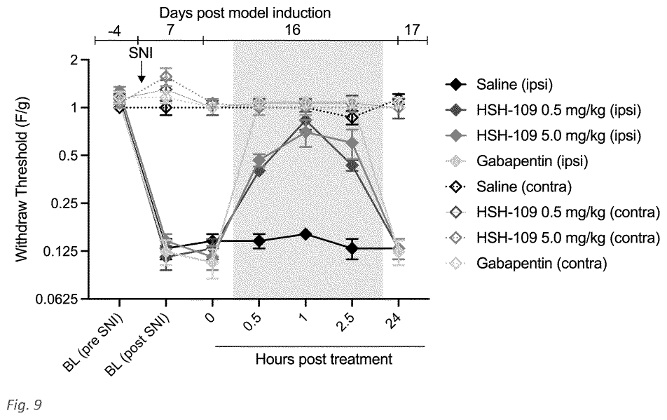

Von Frey filaments ranging from 0.04 to 2 g (g=gram-forces) (0.04, 0.07, 0.16, 0.4, 0.6, 1.0, 1.4, 2.0) were used for determination of mechanical paw withdrawal threshold (PWT). Filaments in ascending order were applied to the frontocentral plantar surface of the hind paws. Mice were placed in red PVC plastic cylinders (8 (∅)×7.5 (h) cm) on a wire mesh and allowed minimum 20 min habituation to the equipment prior of experiment initiation. Each von Frey hair was applied five times with adequate resting periods between each application and number of withdrawals recorded. The withdrawal threshold was determined as the von Frey filament eliciting at least 3 positive trials out of the 5 applications in two consecutive filaments. A positive trial was defined as sudden paw withdrawal, flinching and/or paw licking induced by the filament. Animals were habituated to the experimental room for a minimum of 60 min before initiation of the experiment and the experimenter (female) was blinded to treatment groups. Measurements were performed four days prior to SNI surgery, seven days after SNI surgery, and 16-17 days after SNI surgery (on days with treatment) as indicated in FIG. 9.

### Intraperitoneal Injections

The injection site (left lower abdominal quadrant) was wiped with 2% chlorhexidine. Mice were manually restrained, abdomen side up, with cranial end pointed down. With an angle of 15° to 20°, the abdominal cavity was punctured to inject either 200 μl of indicated doses of indicated compounds, or vehicle (normal saline or PBS, as indicated). Before injection, aspiration was attempted to ensure that an abdominal viscus had not been penetrated.

Data from this setup is presented in FIG. 9. The inventors observed a very robust reduction of mechanical hypersensitivity at both 0.5 mg/kg and 5 mg/kg, suggesting that 0.5 mg/kg is a saturating dose in this setup.

### Example 5—Model of Inflammatory Pain (Complete Freund's Adjuvant, CFA)

The purpose of this experiment was to determine the effect of compounds in a model of inflammatory pain (complete Freund's adjuvant, CFA) as measured by mechanical hypersensitivity.

### Induction of Inflammatory Pain

Injury was induced on the right hind paw, whereas the contralateral left hind paw was used as internal control of the animal. The inflammatory pain was induced by disinfection with EtOH followed by injection of 50 μL undiluted Complete Freund's Adjuvant (CFA) unilaterally into the intraplantar surface of the right hind paw with an insulin needle (0.3 mL BD Micro-Fine). During injections, animals were under isoflurane anesthesia (2%) for maximum 60 seconds.

### Intraperitoneal Injections

The injection site (left lower abdominal quadrant) was wiped with 2% chlorhexidine. Mice were manually restrained, abdomen side up, with cranial end pointed down. With an angle of 15° to 20°, the abdominal cavity was punctured to inject either 200 μl of indicated doses of indicated compounds, or vehicle (normal saline or PBS, as indicated). Before injection, aspiration was attempted to ensure that an abdominal viscus had not been penetrated.

### Measurement of Tactile Sensory Thresholds

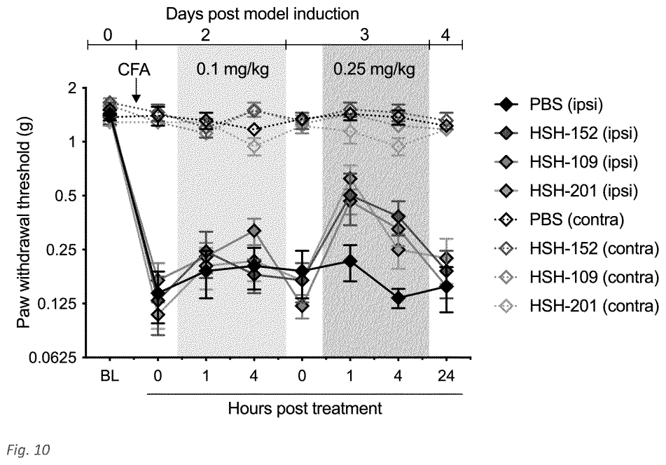

Von Frey filaments ranging from 0.04 to 2 g (g=gram-forces) (0.04, 0.07, 0.16, 0.4, 0.6, 1.0, 1.4, 2.0) were used for determination of mechanical paw withdrawal threshold (PWT). Filaments in ascending order were applied to the frontocentral plantar surface of the hind paws. Mice were placed in red PVC plastic cylinders (8 (∅)×7.5 (h) cm) on a wire mesh and allowed minimum 20 min habituation to the equipment prior of experiment initiation. Each von Frey hair was applied five times with adequate resting periods between each application and number of withdrawals recorded. The withdrawal threshold was determined as the von Frey filament eliciting at least 3 positive trials out of the 5 applications in two consecutive filaments. A positive trial was defined as sudden paw withdrawal, flinching and/or paw licking induced by the filament. Animals were habituated to the experimental room for a minimum of 60 min before initiation of the experiment and the experimenter (female) was blinded to treatment groups. Measurements were performed immediately prior to CFA injection, and on days 2-4 (days 2 and 3 with treatment) at time points indicated in FIG. 10.

Data from this setup is presented in FIG. 10. The inventors observed a reduction of mechanical hypersensitivity at 0.25 mg/kg but not a 0.1 mg/kg. Further, the inventors observed a reduction of mechanical hypersensitivity not only for HSH-109, but also for the analogs HSH-152 and HSH-201.

### Example 6—Biomarkers of Inflammation

The purpose of this experiment was to determine the effect of HSH-109 in reducing the release of inflammatory markers tumor necrosis factor (TNFα) and monocyte chemoattractant protein 1 (MCP1) following induction of lipopolysaccharide (LPS)-stimulated inflammation in mouse macrophages.

### Macrophage Treatment

Peritoneal macrophages were obtained from BALB/c mice and seeded in 24-well plates at 1E5 cells/well in 0.5 mL media. After overnight incubation, 10 nM of HSH-109 and 1 μg/ml lipopolysaccharides (LPS) from Escherichia coli were added. Supernatants were collected 24 h after incubation and stored in −20° C. for cytokine analysis.

### ELISA Measurement of Inflammatory Markers

The plate was coated with 50 μL of appropriate capture antibody (BD 554640 for TNFα, PEP.500-P113 for MCP1) and incubated at 4° C., overnight. The unbound antibody was removed and washed with 200 μL PBS with 0.05% Tween 20 (washing solution). Blocking solution (PBS with 10% FBS) was added and incubated for 2.5 h at room temperature (RT). The blocking solution was removed and 70 μL of the standard or the samples (supernatants) were added. Standards (0.07 to 10 ng/ml) were prepared in blocking solution. After overnight incubation at 4° C., the antibodies were washed four times with washing solution. 100 μL of the appropriate biotin-labeled detection antibody (BD 554415 for TNFα, PEP.500-P113Bt for MCP1) in blocking solution was added to the plate and incubated for 1 h at RT. The wells were washed six times with washing solution. 100 μL of avidin peroxidase was added to each well and incubated for 45 min at RT. The wells were washed 8 times before adding 100 μL 2,2′-azino-bis(3-ethylbenzothiazoline-6-sulfonic acid) (ABTS). Absorbance was read at 405 nm using a Molecular Devices (Spectramax) plate reader.

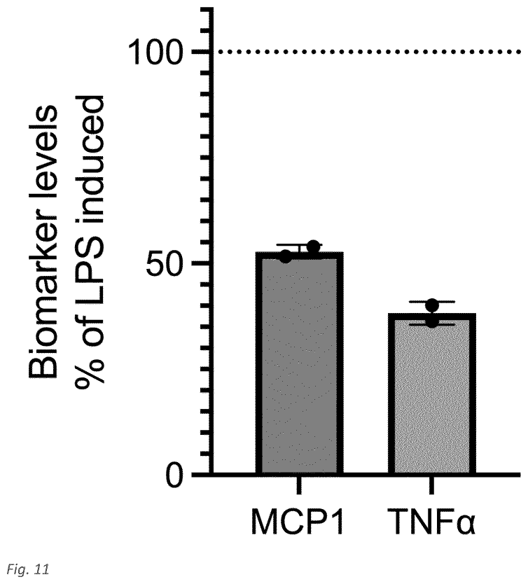

Data from this setup is presented in FIG. 11. The inventors observed that the 10 nM HSH-109 treatment reduced the TNFα levels by ˜62% and the MCP1 levels by ˜47%.

### Example 7—Comparison of Cont-Ro1 and HSH-109 in Post-Surgical Pain Model

The purpose of this was to directly compare the in vivo effect of 2.5 mg/kg Cont-Ro1 and HSH-109 in a post-surgical pain model.

Both experiments were performed in the same lab according to FIG. 5 (HSH-109 data is a normalized data from 2.5 mg/kg from FIG. 5), and the experiments were performed as described in Example 5. Data is normalized to showing percentage of pre-surgery thresholds of force applied using von Frey hairs.

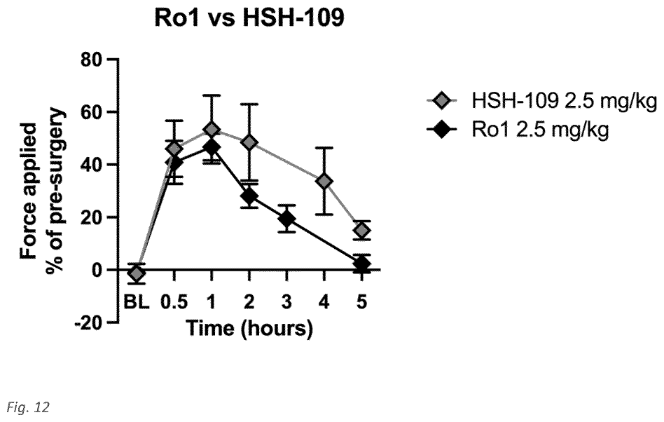

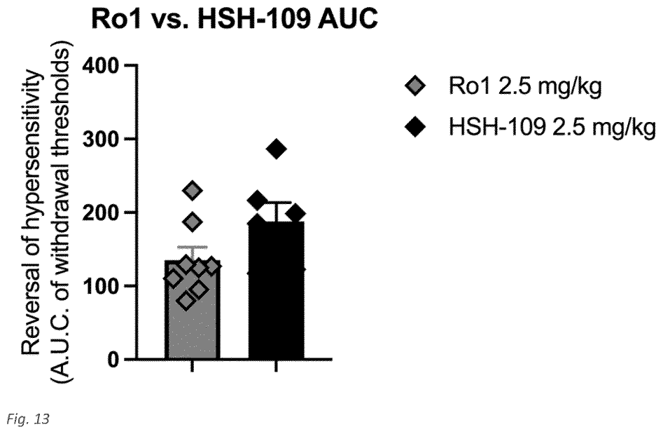

Data from this setup is presented in FIGS. 12 and 13. The inventors observed a tendency for a higher effect level of HSH-109 over Cont-Ro1, which was also longer lasting (FIG. 12). These effects combined result in the area under the curve (FIG. 13) of HSH-109 being 39% larger than for Cont-Ro1. Given the effect of HSH-109 in the SNI model, it is expected to be fully efficacious at 0.5 mg/kg. However, as observed in Ramiro et al., 2022 (Ramiro I B L, Bjørn-Yoshimoto W E, Imperial J S, Gajewiak J, Salcedo P F, Watkins M, Taylor D, Resager W, Ueberheide B, Bräuner-Osborne H, et al. 2022. Somatostatin venom analogs evolved by fish-hunting cone snails: From prey capture behavior to identifying drug leads. Sci Adv. 8(12):eabk1410.), even 1 mg/kg of Cont-Ro1 was not effective at eliciting a robust response.

### Sequence Listing

### ST.25 Standard Definition

(source WIPO, https://www.wipo.int/export/sites/www/standards/en/pdf/archives/03-25-01arc2009.pdf)

According to the ST.25 standard definition, “amino acids” are those L-amino acids commonly found in naturally occurring proteins and are listed in Table 1. Those amino acid sequences containing at least one D-amino acid are not intended to be embraced by this definition. Any amino acid sequence that contains post-translationally modified amino acids may be described as the amino acid sequence that is initially translated using the symbols shown in Table 2, with the modified positions, for example, hydroxylations or glycosylations. But these modifications shall not be shown explicitly in the amino acid sequence. Any peptide that can be expressed as a sequence using the symbols in Table 1, in conjunction with a description elsewhere to describe, for example, abnormal linkages, cross-links (for example, disulfide bridge) and end caps, non-peptidyl bonds, etc., is embraced by the ST.25 standard definition.

TABLE 1
List of amino acids

| | L- three | D- three | L-one | D-one |
|---|---|---|---|---|
| | letter | letter | letter | letter |
| Amino acid | code | code | code | code |
| Alanine | Ala | ala | A | a |
| Cysteine | Cys | cys | C | c |
| Aspartic Acid | Asp | asp | D | d |
| Glutamic Acid | Glu | glu | E | e |
| Phenylalanine | Phe | phe | F | f |
| Glycine | Gly | gly | G | g |
| Histidine | His | his | H | h |
| Isoleucine | Ile | ile | I | i |
| Lysine | Lys | lys | K | k |
| Leucine | Leu | leu | L | l |
| Methionine | Met | met | M | m |
| Asparagine | Asn | asn | N | n |
| Proline | Pro | pro | P | p |
| Glutamine | Gln | gln | Q | q |
| Arginine | Arg | arg | R | r |
| Serine | Ser | ser | S | s |
| Threonine | Thr | thr | T | t |
| Valine | Va | val | V | v |
| Tryptophan | Trp | trp | W | w |
| Tyrosine | Tyr | tyr | Y | y |
| Asp or Asn | Asx | asx | B | b |
| Glu or Gln | Glx | glx | Z | z |
| unknown or other | Xaa | xaa | X | x |

f = D-phenylalanine (D-Phe, D-phenylalanine is the D-enantiomer of phenylalanine)
w = D-tryptophan (D-Trp, D-tryptophan is the D-enantiomer of tryptophan)
O = Hydroxyproline

TABLE 2

| | | | Amino acid |
|---|---|---|---|
| | | | sequence of |
| | | | peptides of the |
| | | | invention |
| | SEQ | ST.25-sequence | (post-translationally |
| Designated name(s) | ID NO. | format | modified) |
| HSH-109 | SEQ ID | PVCWKFGCPL | PVCwKFGCOL |
| | NO. 1 | | |
| HSH-133 | SEQ ID | LFCWKFGCW | LFCwKFGCW |
| (HSH-109-[P1L, V2F, O9W, ΔL10]) | NO. 2 | | |
| HSH-142 | SEQ ID | PVCWKFGCW | PVCwKFGCW |
| (HSH-109-[O9W, ΔL10]) | NO. 3 | | |
| HSH-153 | SEQ ID | PVCWKFACPL | PVCwKFACOL |
| (HSH-109-[G7A]) | NO. 4 | | |
| HSH-141 | SEQ ID | LFCWKFGCPL | LFCwKFGCOL |
| (HSH-109-[P1L, V2F]) | NO. 5 | | |
| HSH-149 | SEQ ID | PVCWKFCPL | PVCwKFCOL |
| (HSH-109-[ΔG7]) | NO. 6 | | |
| HSH-147 | SEQ ID | PVCWKFGCPL | PVCwKFGCPL |
| (HSH-109-[O9P]) | NO. 7 | | |
| HSH-143 | SEQ ID | PVCWKFGCPL | PVCwKfGCOL |
| (HSH-109-[F6f]) | NO. 8 | | |
| HSH-148 | SEQ ID | PVCWKFGCPL | PVCWKFGCOL |
| (HSH-109-[w4W]) | NO. 9 | | |
| HSH-146 | SEQ ID | PVCWKFGC | PVCwKFGC |
| (HSH-109-[ΔO9, ΔL10]) | NO. 10 | | |
| HSH-144 | SEQ ID | CWKFGC | CwKFGC |
| (HSH-109-[ΔP1, ΔV2, ΔO9, ΔL10]) | NO. 11 | | |
| HSH-145 | SEQ ID | CWKFGCPL | CwKFGCOL |
| (HSH-109-[ΔP1, ΔV2]) | NO. 12 | | |
| HSH-152 | SEQ ID | PVCWKAGCPL | PVCwKAGCOL |
| (HSH-109-[F6A]) | NO. 13 | | |
| HSH-151 | SEQ ID | PVCWAFGCPL | PVCwAFGCOL |
| (HSH-109-[K5A]) | NO. 14 | | |
| HSH-140 | SEQ ID | EGYKCWKFGCMPA | yGYKCwKFGCMOA |
| | NO. 15 | | |
| HSH-183 | SEQ ID | WCWKFTCW | WCwKFTCW |
| | NO. 16 | | |
| HSH-187 | SEQ ID | PVCYKFGCPL | PVCyKFGCOL |
| | NO. 17 | | |
| HSH-188 | SEQ ID | PVCHKFGCPL | PVChKFGCOL |
| | NO. 18 | | |
| HSH-200 | SEQ ID | PVCWKFSCW | PVCwKFSCW |
| | NO. 19 | | |
| HSH-201 | SEQ ID | PVCWKFTCW | PVCwKFTCW |
| | NO. 20 | | |
| HSH-202 | SEQ ID | PVCWKFNCW | PVCwKFNCW |
| | NO. 21 | | |
| HSH-203 | SEQ ID | KVCWKFGCPL | KVCwKFGCOL |
| | NO. 22 | | |
| HSH-204 | SEQ ID | MVCWKFGCPL | MVCwKFGCOL |
| | NO. 23 | | |
| HSH-205 | SEQ ID | PVCWKFGCPL | OVCwKFGCOL |
| | NO. 24 | | |
| HSH-206 | SEQ ID | PDCWKFGCPL | PDCwKFGCOL |
| | NO. 25 | | |
| HSH-207 | SEQ ID | PPCWKFGCPL | PPCwKFGCOL |
| | NO. 26 | | |
| HSH-208 | SEQ ID | PPCWKFGCPL | POCwKFGCOL |
| | NO. 27 | | |
| HSH-210 | SEQ ID | PVCFKFGCPL | PVCfKFGCOL |
| | NO. 28 | | |
| HSH-212 | SEQ ID | PVCWRFGCPL | PVCwRFGCOL |
| | NO. 29 | | |
| HSH-213 | SEQ ID | PVCWKFTCPL | PVCwKTGCOL |
| | NO. 30 | | |
| HSH-214 | SEQ ID | PVCWKFSCPL | PVCwKFSCOL |
| | NO. 31 | | |
| HSH-215 | SEQ ID | PVCWKFVCPL | PVCwKFVCOL |
| | NO. 32 | | |
| HSH-216 | SEQ ID | PVCWKFNCPL | PVCwKFNCOL |
| | NO. 33 | | |
| HSH-217 | SEQ ID | PVCWKFLCPL | PVCwKFLCOL |
| | NO. 34 | | |
| HSH-218 | SEQ ID | PVCWKFICPL | PVCwKFICOL |
| | NO. 35 | | |
| HSH-219 | SEQ ID | PVCWKFGCLL | PVCwKFGCLL |
| | NO. 36 | | |
| HSH-220 | SEQ ID | PVCWKFGCIL | PVCwKFGCIL |
| | NO. 37 | | |
| HSH-221 | SEQ ID | PVCWKFGCPP | PVCwKFGCOP |
| | NO. 38 | | |

<110> University of Copenhagen
<120> Novel SST4 selective agonists as non-opioid
analgesics
<130> 78230EP01
<160> 14
<170> BISSAP 1.3.6
<210> 1
<211> 10
<212> PRT
<213> Cone snail
<223> HSH-109
<400> 1
Pro Val Cys Trp Lys Phe Gly Cys Pro Leu
1 5 10
<210> 2
<211> 9
<212> PRT
<213> Cone snail
<223> HSH-133
<400> 2
Leu Phe Cys Trp Lys Phe Gly Cys Trp
1 5
<210> 3
<211> 9
<212> PRT
<213> Cone snail
<223> HSH-142
<400> 3
Pro Val Cys Trp Lys Phe Gly Cys Trp
1 5
<210> 4
<211> 10
<212> PRT
<213> Cone snail
<223> HSH-153
<400> 4
Pro Val Cys Trp Lys Phe Ala Cys Pro Leu
1 5 10
<210> 5
<211> 10
<212> PRT
<213> Cone snail
<223> HSH-141
<400> 5
Leu Phe Cys Trp Lys Phe Gly Cys Pro Leu
1 5 10
<210> 6
<211> 9
<212> PRT
<213> Cone snail
<223> HSH-149
<400> 6
Pro Val Cys Trp Lys Phe Cys Pro Leu
1 5
<210> 7
<211> 10
<212> PRT
<213> Cone snail
<223> HSH-147
<400> 7
Pro Val Cys Trp Lys Phe Gly Cys Pro Leu
1 5 10
<210> 8
<211> 10
<212> PRT
<213> Cone snail
<223> HSH-143
<400> 8
Pro Val Cys Trp Lys Phe Gly Cys Pro Leu
1 5 10
<210> 9
<211> 10
<212> PRT
<213> Cone snail
<223> HSH-148
<400> 9
Pro Val Cys Trp Lys Phe Gly Cys Pro Leu
1 5 10
<210> 10
<211> 8
<212> PRT
<213> Cone snail
<223> HSH-146
<400> 10
Pro Val Cys Trp Lys Phe Gly Cys
1 5
<210> 11
<211> 6
<212> PRT
<213> Cone snail
<223> HSH-144
<400> 11
Cys Trp Lys Phe Gly Cys
1 5
<210> 12
<211> 8
<212> PRT
<213> Cone snail
<223> HSH-145
<400> 12
Cys Trp Lys Phe Gly Cys Pro Leu
1 5
<210> 13
<211> 10
<212> PRT
<213> Cone snail
<223> HSH-152
<400> 13
Pro Val Cys Trp Lys Ala Gly Cys Pro Leu
1 5 10
<210> 14
<211> 10
<212> PRT
<213> Cone snail
<223> HSH-151
<400> 14
Pro Val Cys Trp Ala Phe Gly Cys Pro Leu
1 5 10
<210> 15
<211> 13
<212> PRT
<213> Cone snail
<223> HSH-140
<400> 15
Glu Gly Tyr Lys Cys Trp Lys Phe Gly Cys Met Pro Ala
1 5 10
<210> 16
<211> 8
<212> PRT
<213> Cone snail
<223> HSH-183
<400> 16
Trp Cys Trp Lys Phe Thr Cys Trp
1 5
<210> 17
<211> 10
<212> PRT
<213> Cone snail
<223> HSH-187
<400> 17
Pro Val Cys Tyr Lys Phe Gly Cys Pro Leu
1 5 10
<210> 18
<211> 10
<212> PRT
<213> Cone snail
<223> HSH-188
<400> 18
Pro Val Cys His Lys Phe Gly Cys Pro Leu
1 5 10
<210> 19
<211> 9
<212> PRT
<213> Cone snail
<223> HSH-200
<400> 19
Pro Val Cys Trp Lys Phe Ser Cys Trp
1 5
<210> 20
<211> 9
<212> PRT
<213> Cone snail
<223> HSH-201
<400> 20
Pro Val Cys Trp Lys Phe Thr Cys Trp
1 5
<210> 21
<211> 9
<212> PRT
<213> Cone snail
<223> HSH-202
<400> 21
Pro Val Cys Trp Lys Phe Asn Cys Trp
1 5
<210> 22
<211> 10
<212> PRT
<213> Cone snail
<223> HSH-203
<400> 22
Lys Val Cys Trp Lys Phe Gly Cys Pro Leu
1 5 10
<210> 23
<211> 10
<212> PRT
<213> Cone snail
<223> HSH-204
<400> 23
Met Val Cys Trp Lys Phe Gly Cys Pro Leu
1 5 10
<210> 24
<211> 10
<212> PRT
<213> Cone snail
<223> HSH-205
<400> 24
Pro Val Cys Trp Lys Phe Gly Cys Pro Leu
1 5 10
<210> 25
<211> 10
<212> PRT
<213> Cone snail
<223> HSH-206
<400> 25
Pro Asp Cys Trp Lys Phe Gly Cys Pro Leu
1 5 10
<210> 26
<211> 10
<212> PRT
<213> Cone snail
<223> HSH-207
<400> 26
Pro Pro Cys Trp Lys Phe Gly Cys Pro Leu
1 5 10
<210> 27
<211> 10
<212> PRT
<213> Cone snail
<223> HSH-208
<400> 27
Pro Pro Cys Trp Lys Phe Gly Cys Pro Leu
1 5 10
<210> 28
<211> 10
<212> PRT
<213> Cone snail
<223> HSH-210
<400> 28
Pro Val Cys Phe Lys Phe Gly Cys Pro Leu
1 5 10
<210> 29
<211> 10
<212> PRT
<213> Cone snail
<223> HSH-212
<400> 29
Pro Val Cys Trp Arg Phe Gly Cys Pro Leu
1 5 10
<210> 30
<211> 10
<212> PRT
<213> Cone snail
<223> HSH-213
<400> 30
Pro Val Cys Trp Lys Phe Thr Cys Pro Leu
1 5 10
<210> 31
<211> 10
<212> PRT
<213> Cone snail
<223> HSH-214
<400> 31
Pro Val Cys Trp Lys Phe Ser Cys Pro Leu
1 5 10
<210> 32
<211> 10
<212> PRT
<213> Cone snail
<223> HSH-215
<400> 32
Pro Val Cys Trp Lys Phe Val Cys Pro Leu
1 5 10
<210> 33
<211> 10
<212> PRT
<213> Cone snail
<223> HSH-216
<400> 33
Pro Val Cys Trp Lys Phe Asn Cys Pro Leu
1 5 10
<210> 34
<211> 10
<212> PRT
<213> Cone snail
<223> HSH-217
<400> 34
Pro Val Cys Trp Lys Phe Leu Cys Pro Leu
1 5 10
<210> 35
<211> 10
<212> PRT
<213> Cone snail
<223> HSH-218
<400> 35
Pro Val Cys Trp Lys Phe Ile Cys Pro Leu
1 5 10
<210> 36
<211> 10
<212> PRT
<213> Cone snail
<223> HSH-219
<400> 36
Pro Val Cys Trp Lys Phe Gly Cys Leu Leu
1 5 10
<210> 37
<211> 10
<212> PRT
<213> Cone snail
<223> HSH-220
<400> 37
Pro Val Cys Trp Lys Phe Gly Cys Ile Leu
1 5 10
<210> 38
<211> 10
<212> PRT
<213> Cone snail
<223> HSH-221
<400> 38
Pro Val Cys Trp Lys Phe Gly Cys Pro Pro
1 5 10

### ABBREVIATIONS

ABTS: 2,2′-azino-bis(3-ethylbenzothiazoline-6-sulfonic acid)
AUC: Area under the curve
CFA: Complete Freund's Adjuvant
CNS: Central nervous system
Cont-Ro1: consomatin Ro1 (a cyclical somatostatin receptor activating peptide)
EC₅₀: The concentration that elicits half of the maximal response
ELISA: Enzyme-linked immunosorbent assay
IL: Interleukin
LPS: Lipopolysaccharide
GH: Growth hormone
MCP1: Monocyte chemoattractant protein 1
PRT: Protein
PWT: Paw withdrawal threshold
RT: Room temperature
SNI: Spared nerve injury
SS: Somatostatin
SST: Somatostatin receptor
SST1: Somatostatin receptor 1
SST2: Somatostatin receptor 2
SST3: Somatostatin receptor 3
SST4: Somatostatin receptor 4
SST5: Somatostatin receptor 5
TNFα: Tumor necrosis factor alpha
Xaa: Amino acid at the position indicated by the numeral following

1. A peptide comprising a sequence of the following consecutive amino acids: L-cysteine, D-tryptophan, L-lysine, L-phenylalanine, L-glycine, and L-cysteine, wherein the first indicated cysteine residue is linked to the last indicated cysteine residue via a disulfide bond.

2. A peptide comprising the amino acid sequence selected from the group consisting of SEQ ID NOs 1-17 and SEQ ID NOs 19-38, having the following configurations:
(SEQ ID NO 1)
PVCwKFGCOL,
(SEQ ID NO 2)
LFCwKFGCW,
(SEQ ID NO 3)
PVCwKFGCW,
(SEQ ID NO 4)
PVCwKFACOL,
(SEQ ID NO 5)
LFCwKFGCOL,
(SEQ ID NO 6)
PVCwKFCOL,
(SEQ ID NO 7)
PVCwKFGCPL,
(SEQ ID NO 8)
PVCwKfGCOL,
(SEQ ID NO 9)
PVCWKFGCOL,
(SEQ ID NO 10)
PVCwKFGC,
(SEQ ID NO 11)
CwKFGC,
(SEQ ID NO 12)
CwKFGCOL,
(SEQ ID NO 13)
PVCwKAGCOL,
(SEQ ID NO 14)
PVCwAFGCOL,
(SEQ ID NO 15)
yGYKCwKFGCMOA,
(SEQ ID NO 16)
WCwKFTCW,
(SEQ ID NO 17)
PVCyKFGCOL,
(SEQ ID NO 19)
PVCwKFSCW,
(SEQ ID NO 20)
PVCwKFTCW,
(SEQ ID NO 21)
PVCwKFNCW,
(SEQ ID NO 22)
KVCwKFGCOL,
(SEQ ID NO 23)
MVCwKFGCOL,
(SEQ ID NO 24)
OVCwKFGCOL,
(SEQ ID NO 25)
PDCwKFGCOL,
(SEQ ID NO 26)
PPCwKFGCOL,
(SEQ ID NO 27)
POCwKFGCOL,
(SEQ ID NO 28)
PVCfKFGCOL,
(SEQ ID NO 29)
PVCwRFGCOL,
(SEQ ID NO 30)
PVCwKTGCOL,
(SEQ ID NO 31)
PVCwKFSCOL,
(SEQ ID NO 32)
PVCwKFVCOL,
(SEQ ID NO 33)
PVCwKFNCOL,
(SEQ ID NO 34)
PVCwKFLCOL,
(SEQ ID NO 35)
PVCwKFICOL,
(SEQ ID NO 36
PVCwKFGCLL,
(SEQ ID NO 37)
PVCwKFGCIL
and
(SEQ ID NO 38)
PVCwKFGCOP;
wherein O indicates hydroxyproline;
and wherein the first indicated cysteine residue is linked to the last indicated cysteine residue via a disulfide bond.

3. (canceled)

4. The peptide according to claim 2, wherein the disulfide bond is replaced by a dicarba bond.

5-7. (canceled)

8. An isolated cyclical peptide of SEQ ID NOs 1-38 1-17 and SEQ ID Nos 19-38, having the following configurations:
(SEQ ID NO 1)
PVCwKFGCOL,
(SEQ ID NO 2)
LFCwKFGCW,
(SEQ ID NO 3)
PVCwKFGCW,
(SEQ ID NO 4)
PVCwKFACOL,
(SEQ ID NO 5)
LFCwKFGCOL,
(SEQ ID NO 6)
PVCwKFCOL,
(SEQ ID NO 7)
PVCwKFGCPL,
(SEQ ID NO 8)
PVCwKfGCOL,
(SEQ ID NO 9)
PVCWKFGCOL,
(SEQ ID NO 10)
PVCwKFGC,
(SEQ ID NO 11)
CwKFGC,
(SEQ ID NO 12)
CwKFGCOL,
(SEQ ID NO 13)
PVCwKAGCOL,
(SEQ ID NO 14)
PVCwAFGCOL,
(SEQ ID NO 15)
yGYKCwKFGCMOA,
(SEQ ID NO 16)
WCwKFTCW,
(SEQ ID NO 17)
PVCyKFGCOL,
(SEQ ID NO 19)
PVCwKFSCW,
(SEQ ID NO 20)
PVCwKFTCW,
(SEQ ID NO 21)
PVCwKFNCW,
(SEQ ID NO 22)
KVCwKFGCOL,
(SEQ ID NO 23)
MVCwKFGCOL,
(SEQ ID NO 24)
OVCwKFGCOL,
(SEQ ID NO 25)
PDCwKFGCOL,
(SEQ ID NO 26)
PPCwKFGCOL,
(SEQ ID NO 27)
POCwKFGCOL,
(SEQ ID NO 28)
PVCfKFGCOL,
(SEQ ID NO 29)
PVCwRFGCOL,
(SEQ ID NO 30)
PVCwKTGCOL,
(SEQ ID NO 31)
PVCwKFSCOL,
(SEQ ID NO 32)
PVCwKFVCOL,
(SEQ ID NO 33)
PVCwKFNCOL,
(SEQ ID NO 34)
PVCwKFLCOL,
(SEQ ID NO 35)
PVCwKFICOL,
(SEQ ID NO 36
PVCwKFGCLL,
(SEQ ID NO 37)
PVCwKFGCIL
and
(SEQ ID NO 38)
PVCwKFGCOP;
wherein O indicates hydroxyproline.

9. The isolated cyclical peptide according to claim 8, wherein any tryptophan (W) is D-tryptophan (w) and any proline (P) is hydroxyproline (O).

10. (canceled)

11. The isolated cyclical peptide according to claim 8, for use as a medicament.

12. The isolated cyclical peptide according to claim 8, for use in treatment of pain or inflammation in a human or mammal subject.

13. The isolated cyclical peptide for use according to claim 11, wherein said peptide is administered into the human or mammal subject via an oral, dermal, topical or subcutaneously route.

14. An isolated peptide selected from the group consisting of:
(a) cyclical peptides comprising amino acid sequences set forth in SEQ ID NOs: 1-17 and SEQ ID NOs 19-38, having the following configurations:
(SEQ ID NO 1)
PVCwKFGCOL,
(SEQ ID NO 2)
LFCwKFGCW,
(SEQ ID NO 3)
PVCwKFGCW,
(SEQ ID NO 4)
PVCwKFACOL,
(SEQ ID NO 5)
LFCwKFGCOL,
(SEQ ID NO 6)
PVCwKFCOL,
(SEQ ID NO 7)
PVCwKFGCPL,
(SEQ ID NO 8)
PVCwKfGCOL,
(SEQ ID NO 9)
PVCWKFGCOL,
(SEQ ID NO 10)
PVCwKFGC,
(SEQ ID NO 11)
CwKFGC,
(SEQ ID NO 12)
CwKFGCOL,
(SEQ ID NO 13)
PVCwKAGCOL,
(SEQ ID NO 14)
PVCwAFGCOL,
(SEQ ID NO 15)
yGYKCwKFGCMOA,
(SEQ ID NO 16)
WCwKFTCW,
(SEQ ID NO 17)
PVCyKFGCOL,
(SEQ ID NO 19)
PVCwKFSCW,
(SEQ ID NO 20)
PVCwKFTCW,
(SEQ ID NO 21)
PVCwKFNCW,
(SEQ ID NO 22)
KVCwKFGCOL,
(SEQ ID NO 23)
MVCwKFGCOL,
(SEQ ID NO 24)
OVCwKFGCOL,
(SEQ ID NO 25)
PDCwKFGCOL,
(SEQ ID NO 26)
PPCwKFGCOL,
(SEQ ID NO 27)
POCwKFGCOL,
(SEQ ID NO 28)
PVCfKFGCOL,
(SEQ ID NO 29)
PVCwRFGCOL,
(SEQ ID NO 30)
PVCwKTGCOL,
(SEQ ID NO 31)
PVCwKFSCOL,
(SEQ ID NO 32)
PVCwKFVCOL,
(SEQ ID NO 33)
PVCwKFNCOL,
(SEQ ID NO 34)
PVCwKFLCOL,
(SEQ ID NO 35)
PVCwKFICOL,
(SEQ ID NO 36
PVCwKFGCLL,
(SEQ ID NO 37)
PVCwKFGCIL
and
(SEQ ID NO 38)
PVCwKFGCOP;
wherein O indicates hydroxyproline; or
(b) cyclical peptides having the conserved core sequence consisting of the following consecutive amino acids: L-cysteine, D-tryptophan, L-lysine, L-phenylalanine, L-glycine, and L-cysteine (CwKFGC),
for use as a non-opioid analgesic or an anti-inflammatory agent.
